(19) United States
(12) Patent Application Publication
(10) Pub. No.: US 2007/0276289 A1
(43) Pub. Date: Nov. 29, 2007

(54) Self-contained specimen collection and testing device

(76) Inventor: Paul Hinton, Colton, CA (US)

Correspondence Address: STETINA BRUNDA GARRED & BRUCKER, 75 ENTERPRISE, SUITE 250, ALISO VIEJO, CA, 92656, US

(21) Appl. No.: 11/327,178

(22) Filed: Jan. 6, 2006

(51) Int. Cl. A61B 5/00

(52) U.S. Cl. 600/573

(57) ABSTRACT

An apparatus for collecting and testing a fluid specimen is provided. The apparatus includes a container having a cover which seals the container and a first and a second compartment within the container which are separated by a partition. A temperature sensor is disposed within the first compartment to test the temperature range of the fluid specimen without having to remove the cover. One or more test indicia are disposed within the second compartment and visible through a wall of the container. The test indicia are designed to test for drug residues or other chemical or biological properties in the fluid specimen. The partition has one or more openings located above the level of the sample to be tested to allow the second compartment to be filled with at least a portion of the sample to be tested from the first compartment by tipping the container. As such, the sample may be tested without removing the fluid sample from the container.

### BACKGROUND OF THE INVENTION

1. Field of the Invention

This invention generally relates to devices for collecting a fluid specimen such as urine and testing the specimen for drug residues or other chemicals, and more particularly, to a self-contained specimen collection and testing device that reduces tampering and handling of the specimen.

2. Description of the Related Art

A number of common laboratory diagnostic tests such as drug screening and pregnancy tests utilize urinalysis, which entails collecting and testing urine specimen from individuals. Conventional urinalysis process usually requires an individual to deposit a urine sample in a sample cup and cover the cup with a threaded cap. The sample is then handled by a laboratory technician who removes the cap and inserts one or more test strips into the urine sample to test for drug residues or other chemicals.

In recent years, incidence of tampering with urine specimen taken for drug screening purposes has increased significantly. To reduce tampering and handling of the specimen, a number of self-contained urinalysis devices have been developed. These self-contained testing devices typically consist of a sample cup with test strips disposed around an inner wall of the cup. As soon as the urine sample is deposited into the cup, the fluid wicks into the test strips and reacts with reagents in the test strips, thus immediately producing a test result. While the sample cups with predisposed test strips reduce handling of the fluid specimen, they do not afford the laboratory technician any control over when the test is to be performed. These sample cups are also susceptible to tampering as individuals to be tested could potentially cause substances that would provide a false negative result to wick into the test strips. Additionally, to detect tampering, the technician still has to remove the cap and insert a thermometer into the urine to determine the temperature of the specimen shortly after it is deposited. Thus, there still remains a risk of spilling, contaminating, and tampering of the fluid specimen when using these self-contained testing devices.

In view of the foregoing, there is a need for an improved apparatus for collecting and testing fluid specimen for drugs, drug metabolites, and other chemicals. To this end, there is a particular need for an improved self-contained device for collecting and testing specimen for urinalysis that reduces handling and tampering.

### SUMMARY OF THE INVENTION

In one aspect, the preferred embodiments of the present invention provide an apparatus for testing a fluid specimen such as urine. The apparatus comprises a container having a cover which seals the container. The container has a first volume for receiving a sample of fluid specimen to be tested and a second volume. Preferably, the container also has a partition to separate the first volume from the second volume. A temperature sensor is disposed within the first volume and a plurality of test indicia are disposed within the second volume and visible through a wall of the container. Preferably, the partition comprises an opening located above the level of the sample to be tested to allow the second volume to be filled with at least a portion of the sample to be tested from the first volume by tipping the container, whereby the sample may be tested without removing sample from the container. In one embodiment, the opening is located at least 0.25 inches above the level of the sample to be tested. In certain preferred embodiments, the sample to be tested is urine. In another embodiment, the second volume is substantially enclosed inside the container such that the sample in the first volume will not flow into the second volume when the container is in an upright position. In yet another embodiment, the container has an upper opening, wherein the upper opening has a soft and rounded lip section. Preferably, the container also has a plurality of slots extending along an inner wall of the container, wherein the slots stabilize and secure the partition in a substantially vertical orientation inside the container. In certain preferred embodiments, the container further includes a horizontal piece extending from an inner wall of the container, wherein the horizontal piece engages with the partition to enclose the second compartment. In one implementation, the horizontal piece is pivotable. In another implementation, the horizontal piece has a beveled horizontal edge and the partition has a beveled upper edge, wherein the two edges are configured to engage with each other so as to enclose the second volume.

In another aspect, the preferred embodiments of the present invention provide a method of collecting and testing a urine sample. The method comprises depositing a urine sample into a container, wherein the urine sample is confined to a first section inside the container. The method further comprises sealing the container with a cover, testing the temperature of the urine sample inside the container without removing the cover, transferring at least a portion of the urine sample into a second section inside the container without removing the cover, and testing the portion of the urine sample transferred to the second section for chemical or biological properties without removing the cover. In one embodiment, the portion of the urine sample is transferred from the first section into the second section by tipping the container so that the urine sample can flow from the first section to the second section. In certain preferred implementations, testing the urine sample comprises testing the sample for drug residues. In one embodiment, the urine sample tested comprises human urine.

### BRIEF DESCRIPTION OF THE DRAWINGS

FIG. 1 is a perspective view of a self-contained fluid specimen collection and testing device of one preferred embodiment of the present invention;

FIGS. 2A and 2B schematically illustrate the first and second sides of the partition of the device of FIG. 1;

FIG. 3 is a cross-sectional view of the apparatus of FIG. 1; and

FIG. 4 is a top down view of Section 1-1 of the apparatus shown in FIG. 3.

### DETAILED DESCRIPTION OF PREFERRED EMBODIMENTS

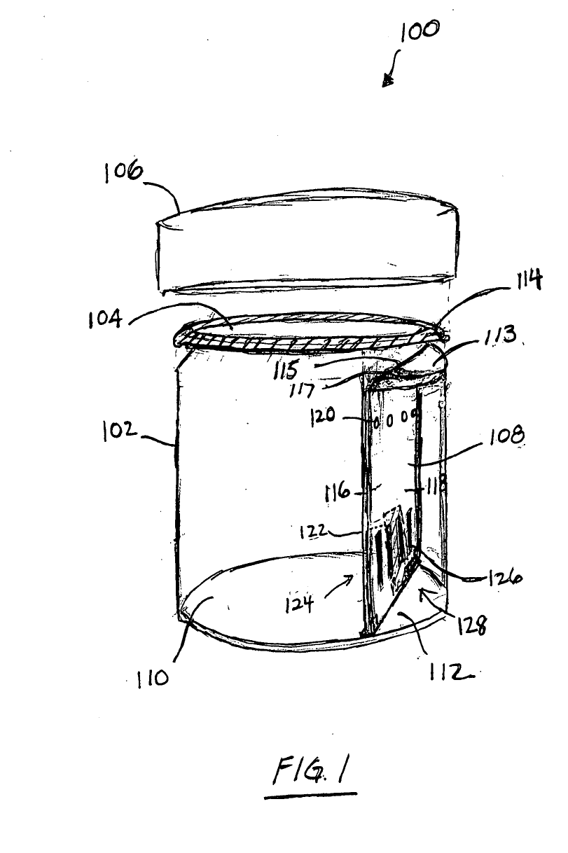

References will now be made to the drawings wherein like numerals refer to like parts throughout. FIG. 1 illustrates a self-contained specimen collection and testing apparatus 100 for sample fluids such as urine. The apparatus 100 generally comprises a container 102 having an upper opening 104 configured to receive a sample of urine or other fluid specimen and a cover 106 that sealingly engages with the upper opening 104 of the container 102. The apparatus 100 further includes a partition 108 which separates the interior of the container 102 into a first-compartment 110 and a second compartment 112. Preferably, the volume of the first compartment 110 is larger than the volume of the second compartment 112.

As will be described in greater detail below, the sample fluid specimen is initially deposited into the first compartment 110 and a portion of the specimen is subsequently transferred into the second compartment 112 when it is ready to be tested. In one embodiment, a horizontal piece 113 extends from an inner wall of the container 102 in a manner such that an outer horizontal edge 115 of the piece 113 meets with an upper horizontal edge 117 of the partition 108 so as to enclose the second compartment 112. The horizontal piece 113 substantially covers the upper opening of the second compartment 112 so as to keep fluid from flowing into the second compartment when it is being deposited into the container 102.

The container 102, the cover 106, and the partition 108 can each be made of a variety of different materials such as plastics or glass. Preferably, the container 102 is made of a substantially clear, lightweight, plastic material and the cover 106 is made of a lightweight, plastic material. Preferably, the cover 102 is a screw lid having threads that engage with corresponding threads on the container. In one embodiment, the container 102 has a smooth and rounded lip portion 114 designed to provide cushion and comfort to individuals depositing urine specimen into the container. The rounded lip portion 114 can be molded as part of the container or can be formed by attaching materials such as plastics, soft rubber, or other polymeric materials to the rim of the container 102.

As further shown in FIG. 1, the partition 108 has two opposing sides 116, 118 and a plurality of openings 120 extending therethrough. The partition 108 is preferably positioned inside the container 102 in a manner such that the first side 116 is adjacent the first compartment 110 and the second side 118 is adjacent the second compartment 112. As will be described in greater detail below, the openings 120 in the partition 108 permit fluid specimen such as urine samples to flow from the first compartment 110 into the second compartment 112 when the container 102 is tilt at an angle, preferably greater than 45 degrees, more preferably greater than 90 degrees.

To thwart tampering, a heat sensor strip 122 is attached to a lower section 124 of the first side 116 of the partition 108 to contact the fluid specimen and test the temperature of the specimen as soon as it is deposited into the container. Preferably, the heat sensor strip 122 is designed to provide a color indicia when the fluid specimen is in the proper temperature range. As shown in FIG. 1, a plurality of chemical test strips 126 are also attached to a lower section 128 of the second side 118 of the partition 108. The chemical test strips 126 are preferably conventional assay strips designed to chemically analyze urine samples for the presence of drug residues in the urine, however they can also include a variety of other known assay test strips. The chemical test strips 126 are positioned to contact and test the fluid specimen transferred from the first compartment 110 to the second compartment 112 through the openings 120 in the partition 108. Since the chemical test strips are enclosed in the second compartment and isolated from the sample fluid, the laboratory technician has full control as to when the test is to be performed. Tampering with the test strips is also substantially reduced as the chemical test strips are inaccessible without taking apart the device.

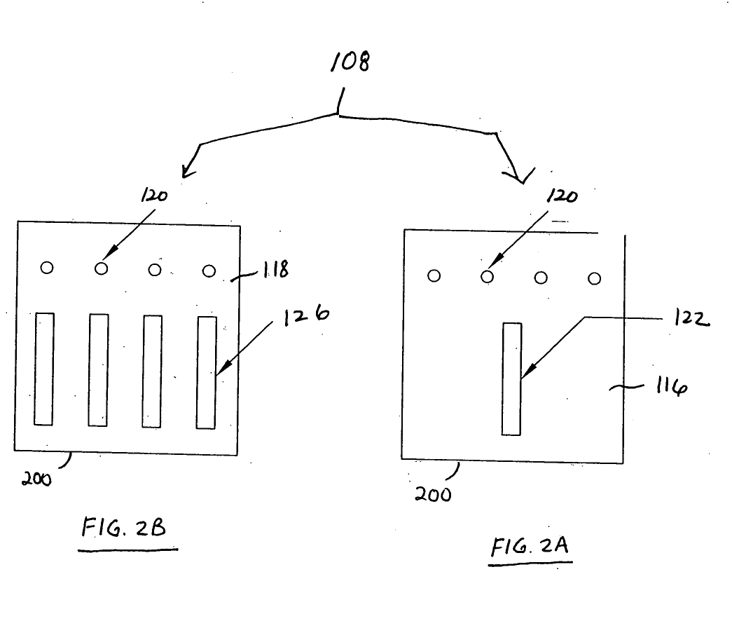

FIGS. 2A and 2B schematically illustrate the first and second sides 116, 118 of the partition 108 of one preferred embodiment of the present invention. The partition 108 is preferably a flat, rectangular, substantially transparent plastic panel. The openings 120 are preferably positioned above the test strips 122, 126 and can assume a variety of different shapes and sizes. In one embodiment, the openings 120 comprise a plurality of circular openings as shown in FIGS. 2A and 2B. In another embodiment, the openings 120 can comprise a single large opening. In another embodiment, the openings 120 are positioned at least 1 inch, preferably 1.5 inch, more preferably 2 inches, from a lower edge 200 of the partition 108.

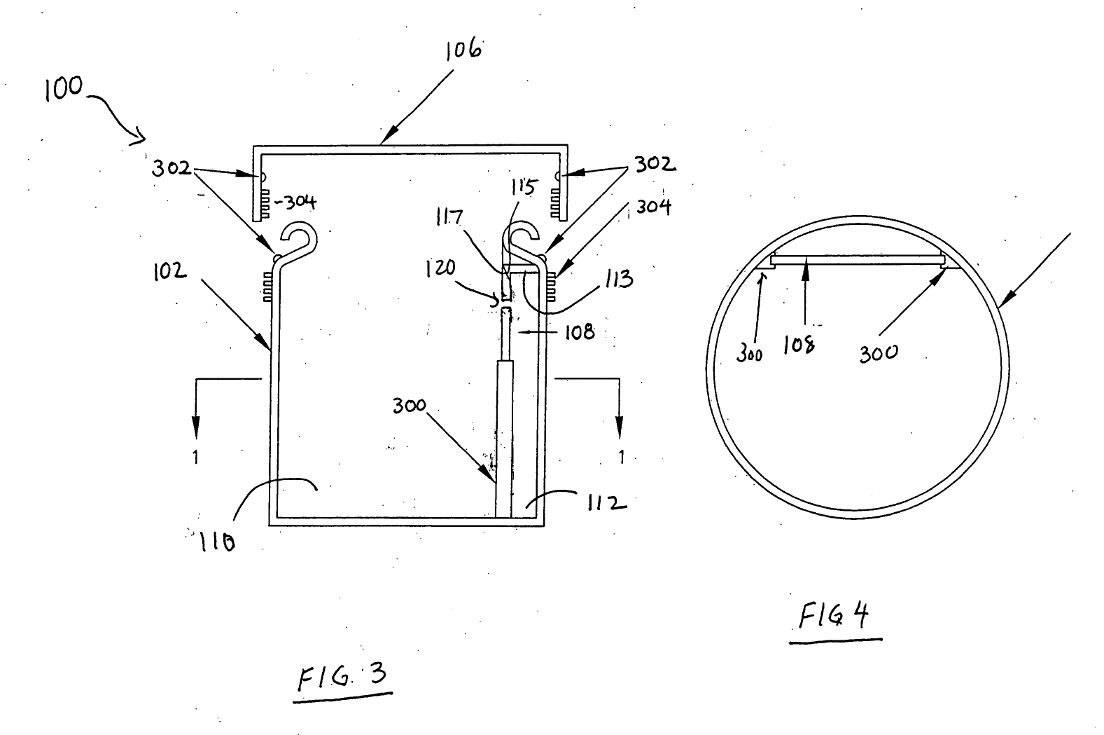

FIG. 3 shows a cross-sectional view of the self-contained specimen collection and testing apparatus 100. As shown in FIG. 3, the partition 108 separates the container 102 into two compartments 110, 112. The two compartments 110, 112 are in fluid communication through openings 120 in the partition 108. As FIG. 3 further shows, the horizontal piece 113 has a beveled edge 115 which is configured to meet the beveled edge 117 on the partition 108 so as to enclose the second compartment 112. The cover 106 and the container 102 have threads 304 that are adapted to engage with each other in a known manner. Seals 302 are also formed on the cover 106 and the container 102 to engage with each other when the cover is placed on the container.

In one embodiment, slotted tabs 300 as shown in FIG. 4 extend along at least a portion of the inner side wall of the container to secure and retain the partition 108 in a substantially vertical orientation inside the container. In another embodiment, the horizontal piece 113 is pivotable such that once the partition 108 is inserted through the slotted tabs 300, the horizontal piece 113 pivots downwardly until the beveled edge 115 of the horizontal piece 113 meets the beveled edge 115 of the partition 108.

In practice, a fluid sample such as urine is deposited into the first compartment of the container such that the level of the fluid is preferably at least 0.25 inch below the opening in the partition. The cover is placed over the container to seal the container. The heat sensing strip on the first side of the partition contacts the fluid and provides an indicia of whether the specimen is within the proper temperature range. When the laboratory technician is ready to test the fluid specimen, the technician tips the container so that fluid flows through the openings in the partition to the second compartment. While in the second compartment, the fluid preferably contacts the chemical testing strips, which test the specimen for drug residues or other chemical and biological properties. Advantageously, there is no need to remove the cover of the apparatus and handle the fluid specimen during the testing process, which reduces contamination and spillage. Additionally, the heat sensing strip in the first compartment automatically tests the temperature of the fluid sample without requiring opening of the container. While the discussions of certain preferred embodiments relate to collecting and testing fluid specimen such as urine from humans, it will be appreciated that the function of the apparatus also extends to collecting and testing fluid specimen from various animals.

Although the foregoing description of the preferred embodiments of the present invention has shown, described and pointed out the fundamental novel features of the invention, it will be understood that various omissions, substitutions, and changes in the form of the detail of the invention as illustrated as well as the uses thereof, may be made by those skilled in the art, without departing from the spirit of the invention. Particularly, it will be appreciated that the preferred embodiments of the invention may manifest itself in other shapes and configurations as appropriate for the end use of the article made thereby.

What is claimed is:

1. An apparatus for collecting and testing a fluid specimen, comprising:
a container having a cover which seals the container, said container having a first volume for receiving a sample of fluid to be tested and a second volume, said container having a partition to separate the first volume from the second volume;
a plurality of test indicia disposed within said second volume and visible through a wall of the container;
a temperature sensor disposed within the first volume;
wherein the partition includes an opening above the level of the sample to be tested to allow the second volume to be filled with at least a portion of the sample to be tested from the first volume by tipping said container, wherein said sample may be tested without removing fluid from the container.

2. The apparatus of claim 1, wherein the second volume is substantially enclosed in the container such that sample in the first volume will not flow into the second volume when the container is in an upright position.

3. The apparatus of claim 1, wherein the container has an upper opening, said upper opening having a soft and rounded lip section.

4. The apparatus of claim 1, wherein said opening is located at least 0.25 inches above the level of the sample to be tested.

5. The apparatus of claim 1, the container further comprises a plurality of slots extending along an inner wall of the container, said slots stabilize and secure the partition in a substantially vertical orientation inside the container.

6. The apparatus of claim 1, wherein the container further comprises a horizontal piece extending from an inner wall of the container, said horizontal piece engages with the partition so as to cover an upper opening of the second volume.

7. The apparatus of claim 6, wherein said horizontal piece is pivotable.

8. The apparatus of claim 1, wherein the partition comprises a substantially rectangular panel.

9. The apparatus of claim 6, wherein said horizontal piece has a beveled horizontal edge and said partition has a beveled upper edge, wherein the two beveled edges engage with each other.

10. The apparatus of claim 1, wherein the temperature sensor provides an indication of the temperature range of the fluid specimen.

11. The apparatus of claim 1, wherein the fluid specimen is urine.

12. A method of collecting and testing a urine sample, comprising:
depositing a urine sample into a container, wherein the urine sample is confined to a first compartment inside the container;
sealing said container with a cover;
testing the temperature of the urine sample inside the container without removing the cover;
transferring at least a portion of the urine sample into a second compartment inside the container without removing the cover; and
testing the portion of the urine sample transferred to the second compartment for chemical or biological properties without removing the cover.

13. The method of claim 12, wherein transferring at least a portion of the urine sample into a second compartment comprises tipping said container so that the urine sample can flow from the first compartment to the second compartment.

14. The method of claim 12, wherein testing the urine sample comprises testing for drug residues.

15. The method of claim 12, wherein the urine sample comprises human urine.
